(19) United States
(12) Patent Application Publication
(10) Pub. No.: US 2008/0255533 A1
(43) Pub. Date: Oct. 16, 2008

(54) Disposable absorbent article with fluid redistribution layer

(75) Inventors: Lanying Zhen Wu, Paoli, PA (US); Linda Morrell-Schwartz, Bensalem, PA (US); Kimberly H. Babusik, Mullica Hill, NJ (US)

Correspondence Address: AMSTER, ROTHSTEIN & EBENSTEIN LLP, 90 PARK AVENUE, NEW YORK, NY, 10016, US

(73) Assignee: Tyco Healthcare Retail Services AG, Schaffhausen (CH)

(21) Appl. No.: 11/787,028

(22) Filed: Apr. 12, 2007

(51) Int. Cl. A61F 13/53

(52) U.S. Cl. 604/385.101

(57) ABSTRACT

A disposable absorbent article having an absorbent core and a fluid redistribution layer disposed on top of the absorbent core, wherein the fluid redistribution layer has a channel which redistributes fluid mainly along the absorbent core. The absorbent core can be disposed between a top sheet and a back sheet of the absorbent article, and the fluid redistribution layer can be disposed between the absorbent core and the top sheet, with the channel having one or more openings on the side next to the top sheet.

### BACKGROUND

The disposable absorbent article with fluid redistribution layer described herein relates generally to disposable absorbent articles, and more particularly to a disposable absorbent article having a core with a fluid redistribution layer for improved fluid handling.

Disposable absorbent articles having absorbent cores and associated fluid acquisition layers, or systems, are known in the art. See, for example, U.S. Pat. Nos. 7,166,094 (Glaug et al.) entitled “Multiple Layer Absorbent Article” and 6,838,591 (Waksmundzki et al.) entitled “Absorbent Article With C-Fold Layer fluid Acquisition System For Use In Absorbent Articles And Method Of Making The Same.”

### SUMMARY

A disposable absorbent article with fluid redistribution layer is described herein, which can generally comprise an absorbent core with the fluid redistribution layer disposed on top of the absorbent core. The fluid redistribution layer can have a generally C-shaped cross section which defines an upwardly opening channel which redistributes fluid along the length of the absorbent core. The absorbent article can further comprise a back sheet and a top sheet, with the absorbent core disposed between the back sheet and the top sheet, and the fluid redistribution layer disposed between the absorbent core and the top sheet. In some embodiments, the fluid redistribution layer can have a generally C-shaped cross section which defines an upwardly opening channel that opens toward the overlying top sheet. The channel can direct fluid away from the center of the absorbent core, such as toward distal ends thereof. In this manner, the fluid can be redistributed over a larger portion of the absorbent core, so that the central portion of the absorbent core does not initially too quickly absorb the fluid, which could cause the disposable absorbent article to leak prematurely. In other embodiments, the fluid redistribution layer can have a rectangular cross section having upper and lower surfaces, and side walls defining the fluid redistribution channel, and one or more openings, such as slots, can be provided through the upper surface into the channel.

Certain illustrative aspects of the disposable absorbent article with fluid redistribution layer are described herein in connection with the following description and the appended drawings. These aspects may be indicative of but a few of the various ways in which the principles of the disposable absorbent article with fluid redistribution layer may be employed, and which is intended to include all such aspects and any equivalents thereof. Other advantages and features of the disposable absorbent article with fluid redistribution layer may become apparent from the following detailed description, when considered in conjunction with the appended drawing figures.

### BRIEF DESCRIPTION OF THE DRAWING FIGURES

A more complete understanding of the disposable absorbent article with fluid redistribution layer can be obtained by considering the following description in conjunction with the accompanying drawings, in which:

FIG. 1 is a top plan view of an embodiment of a disposable absorbent article with fluid redistribution layer.

FIG. 2 is a cross section view taken along line II-II in FIG. 1.

FIG. 3 is a top plan view of another embodiment of a disposable absorbent article with fluid redistribution layer.

FIG. 4 is a cross section view taken along line IV-IV in FIG. 1.

FIGS. 5A and 5B illustrate further embodiments of a fluid redistribution layer.

### DESCRIPTION OF CERTAIN EMBODIMENTS

The terms “absorbent article” or simply “article” refer to devices that absorb and contain body fluids and other body exudates. More specifically, these terms refer to articles that are placed against or in proximity to the body of a wearer to absorb and contain the various exudates discharged from the body. Disposable absorbent articles are well known in the art. A non-exhaustive list of examples of absorbent articles includes diapers, diaper covers, underpads, disposable diapers, training pants, feminine hygiene products and adult incontinence products.

Although a “disposable” absorbent article is described herein, it is to be understood that such “disposable” articles may be intended to be either fully or only partially discarded after a single use. Thus, “disposable” articles can comprise a single inseparable structure, in which the entire article is disposable, or may also comprise articles having replaceable inserts or other interchangeable parts, in which only those inserts or interchangeable parts are disposable.

As used herein, the terms “absorbent article,” “absorbent garment,” or simply “article” or “garment,” refer to devices that absorb and contain body fluids and other body exudates. More specifically, these terms refer to articles that are placed against or in proximity to the body of a wearer to absorb and contain the various exudates discharged from the body. Though different in appearance and dimensions, all of the various types of absorbent articles listed previously can generally perform the same basic function of fluid absorption and retention and can all be generally based upon the same fundamental technology. Nearly all of these types of absorbent articles are comprised of a top sheet, a back sheet, and an absorbent core disposed between the top sheet and back sheet. The absorbent core is conventionally located within the article at a position to receive bodily fluids. Optionally, a fluid acquisition layer may also be disposed between the top sheet and the absorbent core.

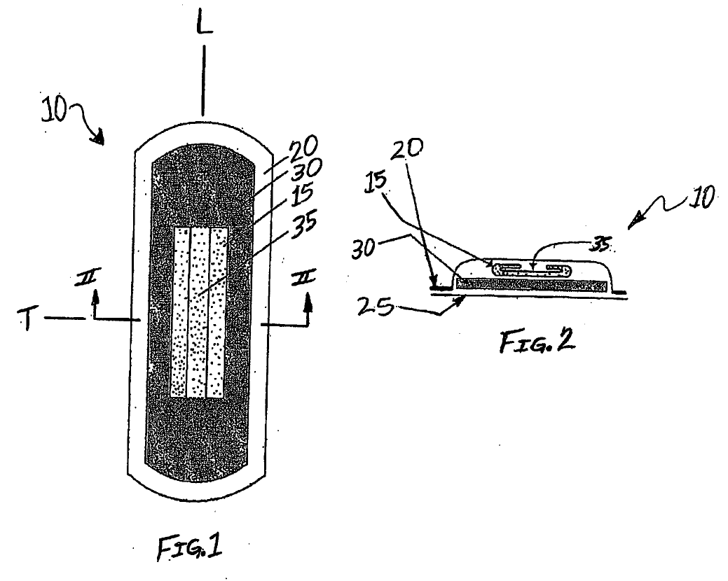

Referring now to the drawing figures, wherein like reference numerals are used to refer to like elements throughout, an embodiment of a disposable absorbent article 10 with fluid redistribution layer 15 is illustrated in the top plan view in FIG. 1. The exemplary embodiment shown can be a Bladder Control Pad (“BCP”), however, this depiction is not intended to limit the various possible embodiments for a disposable absorbent article with the fluid redistribution layer, which can encompass any of the foregoing classes of absorbent articles, without limitation. As used herein, the longitudinal axis L of the article is the dimension of the article corresponding to the front-to-rear dimension of the user, and the transverse axis T is the dimension corresponding to the side-to-side dimension of the user.

Further details of the disposable absorbent article 10 with fluid redistribution layer 15 are illustrated in FIG. 2, which is a cross section view taken along line II-II in FIG. 1. As shown, the disposable absorbent article 10 can further generally comprise a top sheet 20, a back sheet 25, and an absorbent core 30 disposed between at least a portion of the top sheet 20 and at least a portion of the back sheet 25 in a conventional manner. The top sheet 20 can be substantially coterminous with the backsheet 25. When the disposable absorbent article 10 is being worn, the top sheet 20 faces the body of the wearer, whereas the back sheet 25 faces away from the body of the wearer.

The fluid redistribution layer 15 can be disposed between the absorbent core 30 and the top sheet 20, and can define an open channel 35. The channel 35 is open on the side of the fluid redistribution layer 15 opposite the absorbent core 30. The channel 35 can open upwardly toward the overlying top sheet 20 in order to receive discharged fluid into the channel 35 and thereby redirect the fluid along the length of the absorbent core 30. However, the width of the channel 35 can also to some extent redirect fluid along a transverse axis, i.e., the width, of the absorbent core 30, such that the fluid is generally redistributed over both the length and width of the absorbent core 30.

The channel 35 is illustrated as having a length which extends at least partially along the longitudinal axis L of the absorbent core 30, and the fluid redistribution layer is shown having a width less than the width of the absorbent core 30. However, it is to be understood that the channel 35, and/or the overall fluid redistribution layer 15 itself, could be longer and/or wider, depending upon various design considerations.

Although both the absorbent core 30 and the fluid redistribution layer 15 are shown having a generally rectangular shape, other shapes may also be provided such as, for example, oval, dog-bone, hourglass, and others. The shape of the absorbent core 30 and fluid redistribution layer 15 can be selected to provide the greatest absorbency with a reduced amount of material, and can vary depending upon the particular type of disposable absorbent article which is to be produced.

The various parts of the disposable absorbent article 10 are associated with one another to form a structure that preferably maintains its shape during the useful life of the disposable absorbent article 10. As used herein, the term “associated” encompasses configurations whereby a first part is directly joined to a second part by affixing the first part directly to the second part, by indirectly joining the first part to the second part through intermediate members, or by fixing the relative positions of various parts by capturing parts between other parts. Those skilled in the art will appreciate that various methods or combinations of methods may be used to securely join the parts of the disposable absorbent article 10.

The top sheet 20 and back sheet 25 may be constructed from a wide variety of materials known in the art. It is not intended to limit construction to any specific materials for these components. The top sheet 20 and back sheet 25 may be shaped and sized according to the requirements of each of the various types of absorbent article, or to accommodate various user sizes.

The back sheet 25 may be made of any suitable pliable liquid impervious material known in the art or later discovered. Typical back sheet materials include films of polyethylene, polypropylene, polyester, nylon, and polyvinyl chloride and blends of these materials. Other back sheet materials will be readily apparent to those of ordinary skill in the art. The back sheet 25 preferably should have sufficient liquid imperviousness to prevent any leakage of fluids through the back sheet 25. The required level of liquid imperviousness may also vary between different locations on the disposable absorbent article 10. The back sheet 25 may also be made from a laminate of overlaid sheets of material. It is desirable that the back sheet have a degree of air permeability for the comfort of the user. It is not intended to limit the construction to any particular material for the back sheet 25, and other back sheet materials will be readily apparent to those of ordinary skill in the art.

The moisture-permeable top sheet 20 may be made of any suitable relatively liquid-pervious material currently known in the art or later discovered that permits passage of a liquid therethrough. Examples of suitable top sheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 30, and/or provide a comfortable feel to the user. It is not intended to limit the construction to any particular material for the top sheet 20, and other top sheet materials will be readily apparent to those of ordinary skill in the art. Different properties, such as fluid perviousness and hydrophilicity, may be imparted upon the top sheet 20 by treating the top sheet 20 with adhesives, surfactants, or other chemicals, using a composite of different materials, or by other means. The top sheet 20 also may be made from a laminate of overlaid sheets of material. The top sheet 20 also may be treated with skin wellness ingredients like aloe and vitamin E.

The top sheet 20 and back sheet 25 can be substantially coterminous, or they may have different shapes and sizes. The particular design of the top sheet and back sheet may be dictated by manufacturing considerations, cost considerations, and performance considerations, with respect to each of the different classes and types of disposable absorbent articles. The top sheet 20 can be large enough to generally cover the absorbent core 30, and the back sheet 25 can be large enough to prevent leakage from the disposable absorbent article 10. The design of the top sheet 20 and the back sheet 25 is known in the art, and a skilled artisan will be able to produce an appropriate top sheet 20 and an appropriate back sheet 25 without undue experimentation.

The top sheet 20 and the back sheet 25 may be associated with one another using a variety of methods known in the art. For example, they may be thermally, ultrasonically, or chemically bonded to one another. They also may be joined using lines of hot melt adhesive or mechanical fasteners, such as thread, clips, or staples. The particular joining method may be dictated by the types of materials selected for the top sheet 20 and back sheet 25.

The absorbent core 30 can be associated with portions of the top sheet 20, back sheet 25, fluid redistribution layer 15, and/or any other suitable part of the disposable absorbent article 10 by any method known in the art to fix the absorbent core 30 in place. Similarly, the fluid redistribution layer may be associated with the top sheet 20, or the absorbent core 15, or any other suitable part of the disposable absorbent article 10 by any method known in the art, in order to fix the fluid redistribution layer 15 in place with respect to the absorbent core 30.

The absorbent core 30, such as of a conventional BCP for example, can comprise a uniformly distributed layer of pulp and Super Absorbent Polymer (“SAP”). The absorbent core 30 can initially absorb fluid rather quickly because fluid can move into void spaces between pulp fibers and SAP particles. However since the SAP will swell when it absorbs fluid, gel-blocking phenomena can inhibit the fluid from moving toward the ends of the absorbent core 30. As a result, the absorbency rate can be slowed and the disposable absorbent article 10 could start to leak prematurely, before the absorbent core 30 has the opportunity to otherwise reach full absorbent capacity.

However, where the fluid redistribution layer 15 is positioned on top of the absorbent core 30, the fluid can be redistributed by the fluid redistribution layer 15 along the length of the absorbent core 30 in order to facilitate full absorbent capacity, and thus reduce the likelihood of premature leakage. The channel 35 of the fluid redistribution layer 15 can open upwardly toward the top sheet 20 of the absorbent article 10 to create a “pipeline” along the longitudinal axis L of the absorbent core 30 (and also to some degree along the transverse, or lateral, axis thereof) in order to facilitate an initial more rapid redirection of fluid towards the ends of the absorbent core 30. In this manner, discharged fluid can distributed more evenly, and absorbed quickly, over the entire area of the absorbent core 30, as opposed to being initially absorbed primarily into the central portion of the absorbent core 30.

Although described above as being made from pulp and SAP, other examples of suitable materials for use as an absorbent core 30 include tow fibers, creped cellulose wadding, absorbent foams, absorbent sponges, absorbent gelling materials, fiberized cellulose, fluff pulp having tissue or synthetic materials, or any equivalent material or combination of materials. The size and capacity of the absorbent material should correspond to the application, for example, an incontinent brief for an adult may require a larger absorbent core than a diaper for a child. Zoned absorbency also may be used, if desired. For example, more absorbent capacity may be located in particular regions of the disposable absorbent article 10 depending on, for example, the gender of the intended wearer or the intended use for the disposable absorbent article 10. Moreover, the utilization of the fluid redistribution layer 15 described herein can also be taken into account, and further affect, how the absorbent core 30 is constructed and from what materials.

Referring now to FIG. 3, there is shown a partially cut away depiction of another embodiment of a disposable absorbent article 50 with fluid redistribution layer 55 having an open channel 57. The fluid redistribution layer 55 can be disposed over top of an absorbent core 60, in order to redistribute discharged fluid over a wider area of the absorbent core 60. The channel 57 is open on the side of the fluid redistribution layer 55 opposite the absorbent core 60.

This exemplary embodiment can be an infant's diaper, however, other types of disposable absorbent articles may similarly employ a fluid redistribution layer 55. The disposable absorbent article 50 is depicted in a flattened position, with the various elastic components depicted in their extended position for clarity. In the flattened position, the disposable absorbent article 50 generally has an hourglass shaped structure, but it may also have, a rectangular, trapezoidal, or other shape. As used herein, the longitudinal axis L of the disposable absorbent article 50 is the dimension of the disposable absorbent article corresponding to the front-to-rear dimension of the user, and the lateral axis T of the disposable absorbent article is the dimension corresponding to the side-to-side dimension of the user.

In use, the disposable absorbent article 50 is a pant-like garment having a waist-encircling region and a crotch region. The waist-encircling region may comprise a front region 62, corresponding to the front of a wearer's body, and a rear region 64, corresponding to the back of a wearer's body, that are joined together at or near their lateral edges 80, causing the longitudinally distal edges 68 of the disposable absorbent article to form the perimeter of a waist opening. The crotch region 66 extends between the front and rear regions 62, 64, and the crotch edges 70 form the perimeter of a pair of leg openings. The front and rear regions 62, 64 may be joined to one another by permanent seams (not shown) or by releasable fasteners 72. The releasable fasteners 72 may comprise an adhesive tape, a mechanically interlocking fastener, such as a hook and loop fastener 74 or a button or snap, or any other suitable releasable fastening device (e.g., string, twist tie, etc.). The disposable absorbent article also may comprise a target surface 78 associated with a surface of the disposable absorbent article, that is selected to interact with the releasable fasteners 72 to provide them with the grip necessary to hold the disposable absorbent article together. While the embodiment depicted in FIG. 3 shows the releasable fasteners 72 being located in the rear region 64, and the target surface 78 being in the front region 62, skilled artisans will recognize that these locations may be reversed. Additionally, although the embodiment is illustrated as having hook and loop fastener regions 72, 74, those skilled in the art will appreciate that other types of fastening mechanism can also be used. The selection and manufacture of permanent side seams, releasable fasteners 72, and target surfaces 78 is known in the art, and a skilled artisan can produce such structures and devices without undue experimentation.

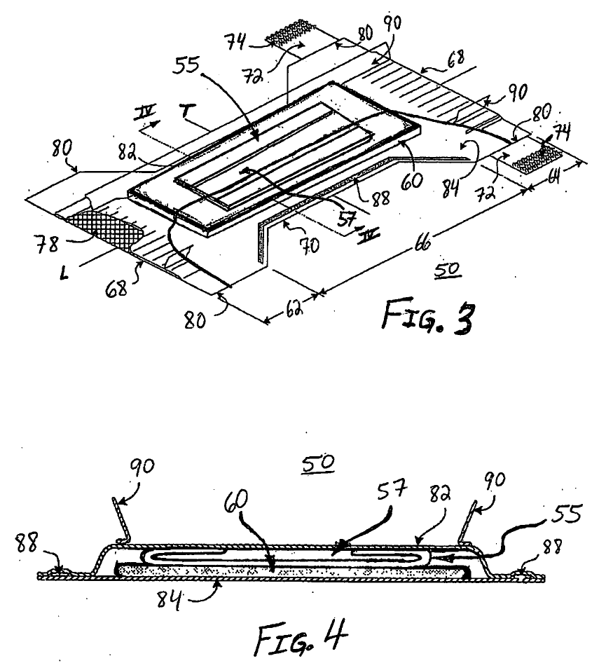

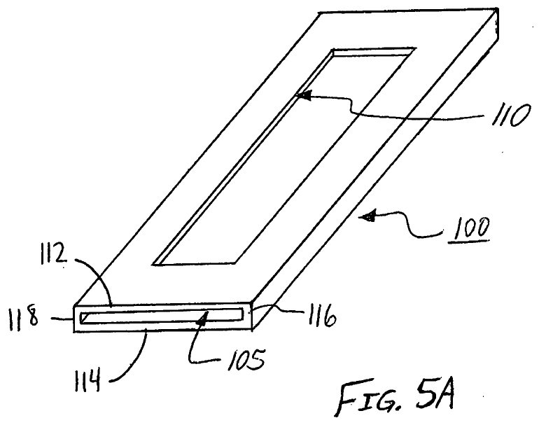

FIG. 4 is a cross-sectional view taken along line IV-IV in FIG. 3, which shows that the disposable absorbent article can comprises a top sheet 82, and a back sheet 84, which may be substantially coterminous with the top sheet 82, with the absorbent core 60 disposed therebetween. When the disposable absorbent article 50 is being worn, the top sheet 82 faces the wearer's body, and the back sheet 84 faces away from the wearer. The fluid redistribution layer 55 can be positioned between the top sheet 82 and the absorbent core 60 with the channel 57 opening upwardly, i.e., toward the top sheet 82.

The fluid redistribution layer 55 can be generally the same shape as the absorbent core 60 and may extend from near the front region 62, through the crotch region 66, and into/near the rear region 64. The fluid redistribution layer 55 can be attached to the absorbent core 60 in any conventional manner. As described previously, the fluid redistribution layer 55 may be somewhat shorter and narrower than the absorbent core 60. However, various different sizes (length and width) of the fluid redistribution layer 55 are possible relative to the size of the absorbent core 60 in order to optimize redistribution of fluid over a larger portion of the absorbent core 60.

Similarly to the previously described embodiment of absorbent core 30 of the disposable absorbent article 10, the absorbent core 60 can be made of pulp fibers and SAP, and thus can similarly absorb fluid rather quickly as the fluid moves into void spaces between pulp fibers and SAP particles. Thus, when the SAP swells as it absorbs fluid, the gel-blocking phenomena can inhibit the fluid from moving toward the ends of the absorbent core 60. As a result, the absorbency rate can be slowed and the disposable absorbent article 50 could start to leak prematurely, before the absorbent core 60 has to opportunity to otherwise reach full absorbent capacity.

However, where the fluid redistribution layer 55 is positioned on top of the absorbent core 60, the fluid can be redistributed by the fluid redistribution layer 55 along the length of the absorbent core 60 in order to facilitate full absorbent capacity, and thus reduce the likelihood of premature leakage. The channel 57 of the fluid redistribution layer 55 can open upwardly toward the top sheet 82 of the absorbent article 50, creating a “pipeline” along the longitudinal axis L of the absorbent core 60 which facilitates an initial more rapid redirection, or redistribution, of fluid towards the ends of the absorbent core 60. In this manner, discharged fluid can be distributed more evenly, and absorbed quickly, over a larger area of the absorbent core 60, as opposed to being initially absorbed primarily into the central portion of the absorbent core 60.

Embodiments of the disposable absorbent article 50 may further comprise various additional features, such as one or more pairs of leg elastics 88 which may extend adjacent the crotch edges 70. The disposable absorbent article 50 also may comprise one or more waste containment systems, such as a pair of standing leg gathers 90. The standing leg gathers 90 preferably extend longitudinally between the front region 62 and the rear region 64 along opposite sides of the disposable absorbent article's longitudinal axis L. The standing leg gathers 90 are shown in FIG. 4 as being comprised of a ribbon-type material disposed above the absorbent core 60. The standing leg gathers 90 may be comprised of different materials, such as a 3-piece top sheet, as is known in the art, and may be disposed anywhere along the top sheet 82, either above the absorbent core 60, on the side edges of the core 60, or laterally outward of the absorbent core 60. The various parts of the disposable absorbent article preferably are associated with one another to form a structure that preferably maintains its shape during the useful life of the disposable absorbent article.

The top sheet 82 and back sheet 84 may be constructed from a wide variety of materials known in the art, and it is not intended to limit the construction of the disposable absorbent article 55, or any components thereof, to any specific materials. Moreover, the top sheet 82 and back sheet 84 can be shaped and sized according to the requirements of each of the various types of disposable absorbent article, or to accommodate various user sizes. In an embodiment in which the disposable absorbent article is a diaper or an adult incontinence brief, the top sheet 82, back sheet 84, or both, may have an hourglass shape, as seen in FIG. 3, or may have a rectangular, trapezoidal, “T” shape, or other shape. In an embodiment in which the disposable absorbent article is a feminine hygiene product, the top sheet 82, back sheet 84, or both, may have a rectangular or oval shape, and may have tabs or “wings.”

The back sheet 84 can generally be made of any suitable pliable liquid impervious material known in the art or later discovered. Typical back sheet materials include films of polyethylene, polypropylene, polyester, nylon, and polyvinyl chloride and blends of these materials. For example, the back sheet 84 may be made of a polyethylene film having a thickness in the range of 0.02-0.04 mm. The back sheet 84 may also be pigmented with, for example, titanium dioxide, calcium carbonate, and other white pigments, to provide the disposable absorbent article with a pleasing color or to render the back sheet 84 opaque enough that exudates being contained by the disposable absorbent article are not visible from outside the disposable absorbent article. In addition, the back sheet 84 may be formed in such a manner that it is opaque, for example, by using various inert components in the polymeric film and then biaxially stretching the film. Other back sheet materials will be readily apparent to those skilled in the art. The back sheet 84 preferably should have sufficient liquid imperviousness to prevent any leakage of fluids through the back sheet 84. The required level of liquid imperviousness may vary between different locations on the disposable absorbent article.

The back sheet 84 may further comprise separate regions having different properties. In some embodiments, portions of the back sheet 84 can be air-permeable to improve the breathability, and therefore comfort, of the disposable absorbent article. The different regions may be formed by making the back sheet 84 of a composite of different sheet materials, chemical treatment, heat treatment, or other processes or methods known in the art. Some regions of the back sheet 84 may be fluid pervious. In some embodiments, the back sheet 84 can be fluid impervious in the crotch 62, but fluid pervious in portions of the front and rear regions 62, 64. The back sheet 84 may also be made from a laminate of overlaid sheets of material.

The back sheet 84 may be covered with a fibrous, non-woven fabric such as is disclosed, for example, in U.S. Pat. No. 4,646,882 issued to Heran et al., which is hereby incorporated by reference in its entirety and in a manner consistent with the present application. Materials for such a fibrous outer liner include a spun-bonded non-woven web of synthetic fibers; a non-woven web of cellulosic fibers, textile fibers, or a blend of cellulosic and textile fibers; a spun-bonded non-woven web of synthetic fibers mixed with cellulosic, pulp fibers, or textile fibers; and melt blown thermoplastic fibers or mixtures of such thermoplastic fibers with cellulosic, pulp or textile fibers. These materials are well known and readily available in the art.

The moisture-pervious top sheet 82 may be made of any suitable relatively liquid-pervious material currently known in the art or later discovered that permits passage of a liquid there through. Examples of suitable top sheet materials include non-woven spun-bonded or carded webs of polypropylene, polyethylene, nylon, polyester and blends of these materials, perforated, apertured, or reticulated films, and the like. Non-woven materials are exemplary because such materials readily allow the passage of liquids to the underlying absorbent core 60.

The top sheet 82 can comprise a single-ply non-woven material that may be made of carded fibers, either adhesively or thermally bonded, perforated or apertured film, spun-bonded fibers, or water entangled fibers, which generally weigh from 0.3-0.7 oz/yd² and have appropriate and effective machine direction and cross-machine (transverse) direction strength suitable for use as a top sheet material for the given application. However, the foregoing examples are not intended to limit the construction to any particular material for the top sheet 82, and other top sheet materials will be readily apparent to those skilled in the art.

The top sheet 82 may further comprise several regions having different properties. In some embodiments, the laterally distal portions of the top sheet 82 can be preferably substantially fluid impervious and hydrophobic, while the remainder of the top sheet 82 can be hydrophilic and fluid pervious. Different top sheet properties, such as fluid perviousness and hydrophobicity, may be imparted upon the top sheet 82 by treating the top sheet 82 with adhesives, surfactants, or other chemicals, using a composite of different materials, or by other methods. Alternatively, the different properties can be achieved by making the top sheet from three separate components, a central, fluid pervious portion, and two lateral fluid impervious portions that can also serve to form standing leg gathers. Such a configuration is described in, for example, U.S. Pat. No. 6,068,670, the disclosure of which is incorporated by reference herein in its entirety. The top sheet 82 also may be made from a laminate of overlaid sheets of material. The top sheet 82 also may be treated in specific areas like the crotch region, with skin wellness ingredients like aloe and vitamin E.

As noted elsewhere herein, the top sheet 82 and back sheet 84 may be substantially coterminous, or they may have different shapes and sizes. The particular design of the top sheet and back sheet may be dictated by manufacturing considerations, cost considerations, and performance considerations. The top sheet 82 can be large enough to completely cover the absorbent core 60, and the back sheet 84 can be large enough to prevent leakage from the disposable absorbent article. The design of top sheets 82 and back sheets 84 is known in the art, and a skilled artisan will be able to produce an appropriate top sheet 82 and an appropriate back sheet 84 without undue experimentation, using the guidelines provided herein.

The top sheet 82 and the back sheet 84 may be associated with one another using a variety of methods known in the art. For example, they may be thermally, ultrasonically, chemically, or thermal mechanically bonded to one another. They also may be joined using lines of hot melt adhesive or mechanical fasteners, such as thread, clips, or staples. In one embodiment, a hydrophilic adhesive, such as CYCLOFLEX™. as sold by National Starch, located in Bridgewater, N.J., is used to join the top sheet 82 to the back sheet 84. The particular joining method may be dictated by the types of materials selected for the top sheet 82 and back sheet 84.

The absorbent core 60 can be disposed between the top sheet 82 and the back sheet 84 in at least the crotch region 66, and can extend into either or both of the front and rear regions 62, 64. Although the absorbent core 60 depicted in FIG. 3 has a substantially rectangular shape, other shapes may be used, such as a “T” shape, an hourglass shape, or other shapes. The shape of the absorbent core 60 may be selected to provide the greatest absorbency with a reduced amount of material. The absorbent core may be associated with the top sheet 82, back sheet 84, or any other suitable part of the disposable absorbent article by any method known in the art, in order to fix the absorbent core 60 in place.

Although the absorbent core 60 is described as being made of pulp and SAP, other examples of suitable materials for use as an absorbent core 60 include tow fibers, creped cellulose wadding, absorbent foams, absorbent sponges, super absorbent polymers, absorbent gelling materials, fiberized cellulose, fluff pulp having tissue or synthetic materials between the absorbent core 60 and the top sheet 82 or any equivalent material or combination of materials. It is not intended to limit the construction to any specific materials for use in the absorbent core 60. Absorbent cores of SAP distributed within a fibrous structure are known in the art, and exemplary absorbent cores are described in U.S. Pat. Nos. 5,801,707, and 6,068,670 issued to Chmielewski et al., and U.S. Pat. No. 5,863,808, issued to Baker, the disclosures of which are herein incorporated by reference in their entirety.

Additional sublayers, transfer layers, acquisition layers, tissue wraps, and the like also may be incorporated into or otherwise associated with the absorbent core 60. Such layers may be provided to assist with transferring fluids to the absorbent core 60, handling fluid surges, preventing rewet, containing absorbent material, improving core stability, or for other purposes. For example, a substantially rectangular, non-woven sublayer (not shown) having a basis weight of about 0.1-2 oz., or of about 0.4-0.6 oz., may overlay absorbent core 60, such as between the absorbent core 60 and the fluid redistribution layer 55. Those skilled in the art are capable of selecting materials, dimensions, and locations for such layers without undue experimentation.

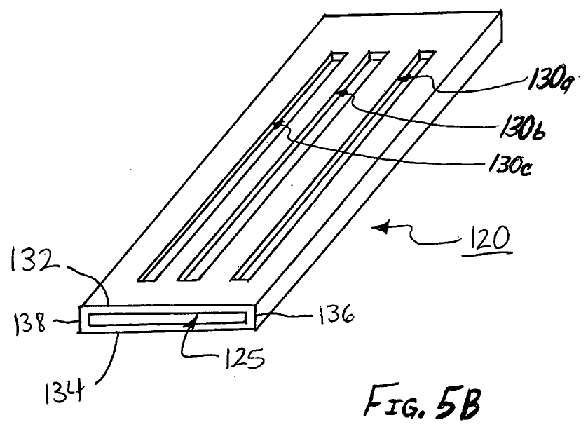

Referring now to FIGS. 5A and 5B, further embodiments of the fluid redistribution layer are shown. In FIG. 5A, the fluid redistribution layer 100 is shown having a channel 105 therethrough and an opening 110 into the channel 105. More particularly, the fluid redistribution layer can have a generally rectangular cross section comprised of an upper surface 112, lower surface 114, and a pair of side walls 116, 118 which define the channel 105. The opening 110, which can be a longitudinally extending slot, can be provided through the upper surface 112 to received fluid into the channel 105 for redirection thereby along the length of an absorbent core associated with the fluid redistribution layer 100.

The embodiment of a fluid redistribution layer 120 shown in FIG. 5B similarly has a channel 125 therethrough, but can have a plurality of openings 130a, 130b, and 130c into the channel 125. The fluid redistribution layer 120 can have a similar generally rectangular cross section comprised of an upper surface 132, lower surface 134, and a pair of side walls 136, 138 which define the channel 125. In this embodiment however, a plurality, three shown for example, of openings 130a, 130b, and 130c (which can also be longitudinally extending slots) can be provided through the upper surface 132 to permit fluid to be received into the channel 125 for redirection thereby along over a larger area of an absorbent core associated with the fluid redistribution layer 120.

Although the openings into the channel have been shown and described as one or more longitudinally extending slots, it will be understood that the openings may take different shapes, in keeping with the main function of such openings being to deliver fluid into the channel for redirection over a larger area of an absorbent core associated with the redistribution layer.

Accordingly, what has been described above includes exemplary embodiments of a disposable absorbent article having a fluid redistribution layer disposed over the absorbent core of the article. It is, of course, not possible to describe every conceivable combination of components or methodologies for purposes of this description, but one of ordinary skill in the art may recognize that further combinations and permutations are possible in light of the overall teaching of this disclosure. Accordingly, the description provided herein is intended to be illustrative only, and should be considered to embrace any and all alterations, modifications, and/or variations that fall within the spirit and scope of the appended claims.

What is claimed is:

1. A disposable absorbent article comprising an absorbent core and a fluid redistribution layer disposed on top of said absorbent core, said fluid redistribution layer defining a channel extending along at least a portion of a longitudinal axis of said absorbent core, and an opening into said channel provided on a side of said fluid redistribution layer opposite said absorbent core.

2. The disposable absorbent article of claim 1 wherein said fluid redistribution layer comprises a C-shaped cross section defining an open channel, and wherein said channel is open on a side of said fluid redistribution layer opposite said absorbent core.

3. The disposable absorbent article of claim 2 further comprising a top sheet, wherein said fluid redistribution layer is disposed between said absorbent core and said top sheet, and said channel opens toward said top sheet.

4. The disposable absorbent article of claim 1 wherein said fluid redistribution layer has a generally rectangular shaped cross section comprising an upper surface, a lower surface and a pair of side walls, said upper and lower surfaces and said pair of side walls defining said channel, and said upper surface having at least one opening into said channel.

5. The disposable absorbent article of claim 4 wherein said at least one opening comprises a longitudinally extending slot.

6. The disposable absorbent article of claim 4 wherein said at least one opening comprises a plurality of longitudinally extending slots.

7. A disposable absorbent article comprising:
a top sheet, a back sheet, and an absorbent core disposed at least partly therebetween;
a fluid redistribution layer disposed between said absorbent core and said top sheet;
said fluid redistribution layer defining a channel extending along at least a portion of said absorbent core; and
at least one opening into said channel on a side thereof adjacent said top sheet, such that fluid is redistributed by said channel over a larger area of said absorbent core.

8. The disposable absorbent article of claim 7 wherein said fluid redistribution layer comprises a C-shaped cross section defining an upwardly opening channel, wherein said channel is open on a side of said fluid redistribution layer adjacent said top sheet.

9. The disposable absorbent article of claim 7 wherein said fluid redistribution layer has a generally rectangular shaped cross section comprising an upper surface, a lower surface and a pair of side walls, said upper and lower surfaces and said pair of side walls defining said channel, and said upper surface having at least one opening into said channel, said at least one opening adjacent said top sheet.

10. The disposable absorbent article of claim 9 wherein said at least one opening comprises a longitudinally extending slot.

11. The disposable absorbent article of claim 10 wherein said at least one opening comprises a plurality of longitudinally extending slots.
